(19) United States
(12) Patent Application Publication
(10) Pub. No.: US 2024/0122517 A1
(43) Pub. Date: Apr. 18, 2024

(54) BIOLOGICAL INFORMATION MEASURING APPARATUS AND BIOLOGICAL INFORMATION PROCESSING SYSTEM

(71) Applicant: OMRON HEALTHCARE CO., LTD., Kyoto (JP)

(72) Inventors: Yasuhiro KAWABATA, Kyoto (JP); Kenji FUJII, Kyoto (JP); Naomi MATSUMURA, Kyoto (JP); Akito ITO, Kyoto (JP); Yuki SAKAGUCHI, Kyoto (JP); Reiji FUJITA, Kyoto (JP); Daizo OKA, Kyoto (JP)

(21) Appl. No.: 18/536,661

(22) Filed: Dec. 12, 2023

Related U.S. Application Data
(63) Continuation of application No. PCT/JP2022/029895, filed on Aug. 4, 2022.

(30) Foreign Application Priority Data
Aug. 4, 2021 (JP) 2021-128376

(51) Int. Cl. A61B 5/282; A61B 5/00; A61B 5/021; A61B 5/022; A61B 5/256; A61B 5/319

(52) U.S. Cl. CPC A61B 5/282; A61B 5/02125; A61B 5/022; A61B 5/256; A61B 5/319; A61B 5/6824; A61B 5/6831; A61B 2562/0204; A61B 2562/0247; A61B 2562/043; A61B 2562/063

(57) ABSTRACT

A biological information measurement device adapted to be attached on an upper arm of a human body and used thereon includes a belt portion adapted to be wound around the upper arm, electrocardiographic measurement means including a plurality of electrodes for detecting an electrocardiographic signal of the human body, pulse wave measurement means including a pulse wave sensor for detecting a pulse wave of the human body, heartbeat vibration measurement means including a vibration sensor for detecting vibration caused by beating of a heart of the human body, and an analysis processing unit configured to calculate a pre-ejection time and a pulse transit time of the heart based on time series data of the electrocardiographic signal, time series data of the pulse wave, and time series data of the vibration caused by the beating of the heart.

### CROSS-REFERENCE TO RELATED APPLICATIONS

This application is the U.S. national stage application filed pursuant to 35 U.S.C. 365(c) and 120 as a continuation of International Patent Application No. PCT/JP2022/029895, filed Aug. 4, 2022, which application claims priority to Japanese Patent Application No. 2021-128376, filed Aug. 4, 2021, which applications are incorporated herein by reference in their entireties.

### TECHNICAL FIELD

The present invention belongs to the technical field related to healthcare and particularly relates to measurement and processing of biological information.

### BACKGROUND ART

In recent years, health management in which information related to the body and health of an individual (hereinafter also referred to as “biological information”) such as a blood pressure value and an electrocardiographic waveform is measured non-invasively by a measurement device and the measurement results are recorded and analyzed by an information processing terminal has been becoming more and more common.

As an example of the measurement device described above, a blood pressure measurement device based on a so-called oscillometric method is widely used. Such a blood pressure measurement device that automatically performs processing of compressing and releasing (the blood vessels of) the upper arm by contraction of a cuff, and of estimating a blood pressure value by measuring pressure in the cuff at that time, does not require special knowledge or skill in operation, making it very easy for a general user to measure blood pressure on a daily basis.

Meanwhile, in recent years, there has been a growing need for early detection of a disease and appropriate treatment performed by constantly wearing a measurement device on the body in daily life and continuously acquiring biological information. However, the conventional blood pressure measurement device using the oscillometric method cannot continuously measure blood pressure. In other words, in the oscillometric method, a procedure of compressing and releasing the upper arm that takes time is required for blood pressure measurement, making it difficult to perform continuous blood pressure measurement (for example, for each heartbeat) in the first place. In addition, since the upper arm is compressed in every blood pressure measurement, it is a large burden for a user to constantly perform such measurement in daily life.

On the other hand, a technique for continuously measuring blood pressure by estimating the blood pressure based on biological information that can be acquired non-invasively at all times by a wearable device has been proposed (for example, Patent Document 1). Patent Document 1 discloses a patch-type biosensor including an electrocardiographic (ECG) sensor, a photoplethysmography (PPG) sensor, a phonocardiogram (PCG) sensor, and the like that are attached to the chest of a human body, with systolic blood pressure (SBP), diastolic blood pressure (DBP), and mean blood pressure (MBP) being predicted based on cardiac timing characteristics such as pulse arrival time (PAT) and pulse transit time (PTT) and PPG signal characteristics that are obtained based on biological signals acquired from the respective sensors. A method of estimating a blood pressure value from a biological indicator such as the PAT or the PTT is known. In particular, when the PTT is used, a shift corresponding to a pre-ejection period (PEP) of the heart can be eliminated, and thus a relatively highly accurate blood pressure value can be predicted. Therefore, according to the technique disclosed in Patent Document 1, a relatively accurate blood pressure value can be acquired non-invasively and continuously at all times only by wearing one sensor device.

### CITATION LIST

### Patent Literature

Patent Document 1: JP 2020-517322 T

### SUMMARY OF INVENTION

### Technical Problem

However, the biosensor device of Patent Document 1 is attached on the chest, and there is a problem that it is difficult to wear the sensor while dressed. Further, the sensor is attached on the chest by adhesion. In such a case, when the sensor is strongly adhered to the chest, invasiveness to the skin increases. Meanwhile, when the adhesive force is weak, the sensor may fall off. Furthermore, from the viewpoint of acquiring a PPG signal, when the PPG sensor is disposed at a position close to the heart, the PAT and the PTT become short. Accordingly, an S/N (signal/noise) ratio (robustness) of the data may decrease. In addition, from the viewpoint of acquiring an accurate ECG signal, a patch needs to be attached such that the positional relationship between an electrode as the ECG sensor and the heart is appropriate, which may cause a problem that a general user who does not have special knowledge has difficulty wearing the sensor device at an appropriate position by themselves.

In view of the above problems, an object of the present invention is to provide a wearable sensor that includes a plurality of biological information sensors and that can be easily attached and detached without burden even by a user who does not have special knowledge, and a technique related to biological information measurement using the wearable sensor.

### Solution to Problem

A biological information measurement device according to the present invention adopts the following configurations in order to solve the above problems. In other words, a biological information measurement device adapted to be attached on an upper arm of a human body and used thereon includes:
a belt portion adapted to be wound around the upper arm;
electrocardiographic measurement means including a plurality of electrodes for detecting an electrocardiographic signal of the human body;
pulse wave measurement means including a pulse wave sensor for detecting a pulse wave of the human body;
heartbeat vibration measurement means including a vibration sensor for detecting vibration caused by beating of a heart of the human body; and
an analysis processing unit configured to calculate a pre-ejection time and a pulse transit time of the heart based on time series data of the electrocardiographic signal, time series data of the pulse wave, and time series data of the vibration caused by the beating of the heart.

In the description above, the “vibration caused by the beating of the heart” can be detected, for example, as heart sounds or heart beating, and a phonocardiogram and a ballistocardiogram (BCG) can be obtained from these time series data. Further, the “pre-ejection time” above is the time from when an electrical signal related to the beating of the heart is detected to when the beating of the heart actually starts (blood is discharged). Furthermore, the “pulse transit time” is the time required for a pulse wave to transit between two different points in the blood vessel.

In addition, in the present specification, for example, information (including a P signal; the same shall apply hereinafter) related to an electrocardiographic waveform (an electrocardiogram), information related to a pulse wave waveform, information related to a phonocardiogram, and information related to a ballistocardiogram may be respectively referred to as ECG data, pulse wave data (PPG data, particularly, in the case of a photoelectric pulse wave), PCG data, and BCG data.

According to the configuration described above, the electrocardiographic signal, the pulse wave, and the vibration caused by the beating of the heart can be detected by one device attached on the upper arm, and the pre-ejection time and the pulse transit time can be calculated. In addition, the device can be attached on the upper arm with a belt, and thus even a user who does not have special knowledge can easily attach and detach the device without burden.

Additionally, the biological information measurement device may further include a first blood pressure measurement unit configured to calculate a blood pressure value of the human body based on the pulse transit time calculated by the analysis processing unit. Since the pulse transit time can be continuously calculated for each heartbeat, such a configuration enables continuous blood pressure measurement at all times based on the pulse transit time.

Moreover, the biological information measurement device may further include: a pressing cuff; fluid supply means configured to supply a fluid to the pressing cuff; a pressure sensor configured to detect pressure in the pressing cuff; and a second blood pressure measurement unit configured to calculate a blood pressure value of the human body based on an output signal of the pressure sensor. For example, a blood pressure measurement method using a cuff, such as an oscillometric method, has established reliability with respect to the accuracy. Therefore, by providing a blood pressure measurement unit using such a measurement method, blood pressure measurement with high accuracy can be performed, and a situation where it is desired to obtain an accurate blood pressure value can be also addressed.

In addition, the first blood pressure measurement unit may perform, based on the blood pressure value measured by the second blood pressure measurement unit, calibration of a calculation formula for calculating a blood pressure value based on the pulse transit time. With such a configuration, calibration (calibration of the calculation formula) of the blood pressure value calculated from the pulse transit time can be performed based on the highly accurate blood pressure measurement result, thereby increasing the accuracy of the blood pressure value calculated based on the pulse transit time.

Moreover, the pulse wave sensor may be disposed to be located on a side closer to a periphery of the human body than the plurality of electrodes, in a state where the biological information measurement device is attached on the upper arm. When obtaining the pulse transit time from the ECG or PCG (or BCG), as a pulse wave detection position becomes farther from the central side of the human body (i.e., the side close to the heart), that is, as the pulse transit time becomes longer, data with a higher S/N ratio can be acquired. Therefore, as described above, by arranging the pulse sensor on the side closer to the periphery (that is, the side farther from the heart), an accurate pulse transit time can be easily calculated.

Further, the biological information measurement device may include a housing in which at least the vibration sensor is housed. The vibration sensor may be housed at a location that is near an inner wall surface of the housing and is located farthest from the skin surface of the human body, in a state where the biological information measurement device is attached on the upper arm. When the vibration caused by the beating of the heart is transmitted through the human body (arm) and shakes the device, amplitude increases as a position becomes farther from the skin surface. Therefore, with such a configuration, (the waveform of) the vibration can be easily detected.

Furthermore, in the biological information measurement device, the vibration sensor may include a plurality of vibration sensors, and the plurality of vibration sensors may be disposed at intervals from a side close to a periphery of the human body to a side close to a center of the human body, in a state where the biological information measurement device is attached on the upper arm. With such a configuration, a noise component can be removed (reduced) by comparing signals output from the plurality of sensors having different transit distances of vibration.

In addition, the vibration sensor may be a sensor mounted on a substrate, and the plurality of vibration sensors may be mounted on different substrates separated from each other. Such a configuration is desirable in order to remove (reduce) the noise component. This is because when the sensors are integrated by a rigid body such as a substrate even though the sensors themselves are separately placed, the same vibration data (signal) is acquired.

Moreover, in the biological information measurement device, the vibration sensor may include a plurality of vibration sensors, and the plurality of vibration sensors may include at least one set of vibration sensors disposed at positions opposed to each other in a circumferential direction of the upper arm, in a state where the biological information measurement device is attached on the upper arm.

In the case of a wearable device that is constantly worn and continuously measures body information, the sensor is compressed and vibration is attenuated depending on the posture of a wearer (for example, in the supine position at bedtime), which may cause a situation where vibration with appropriate intensity cannot be detected. In this regard, the plurality of vibration sensors are disposed at positions opposed to each other with the arm interposed therebetween as described above, and thus at least one of the sensors can detect appropriate vibration.

Further, the vibration sensor may be disposed to be located near an end portion on a side close to a center of the human body, in a state where the biological information measurement device is attached on the upper arm. Since the vibration to be detected by the vibration sensor is due to the beating of the heart, it is desirable that the vibration sensor is disposed on the central side of the human body (i.e., the side close to the heart) as in the configuration described above in order to obtain an appropriate signal.

Furthermore, at least one of the plurality of electrodes and the vibration sensor may be integrally formed, and the vibration sensor may be disposed on a side opposed to a contact surface of the electrode integrally formed with the vibration sensor, the contact surface contacting the human body. At such a position opposed to the contact surface of the electrode, a mechanism for fixing the electrode or a substrate for acquiring potential information of the electrode is usually arranged. However, by providing the vibration sensor integrally with such a mechanism, a dedicated member (substrate or the like) on which the vibration sensor is mounted can be omitted.

In addition, the vibration sensor may be a microphone, and the electrode integrally formed with the vibration sensor may be provided with a sound pickup structure. The electrode and a function to amplify the vibration acquired by the microphone are integrally configured as just described, and thus an S/N ratio of a signal acquired by the vibration sensor can be improved while the number of components is reduced.

Moreover, the sound pickup structure may be a hollow portion disposed to extend through the electrode in a thickness direction. Further, the hollow portion may be filled with a resin having a hardness comparable with a hardness of human skin to be flush with the contact surface. The vibration is acquired through the resin having hardness comparable with that of the skin, and thus transit efficiency of the vibration is improved, and the S/N ratio of the acquired signal can be improved.

Furthermore, the resin may be a conductive resin. With such a configuration, a reduction in contact resistance in the contact surface of the electrode in which the contact area with the skin is reduced by providing the hollow portion can be suppressed.

In addition, the present invention may be also defined as a biological information processing system including:
a sensor device including a belt portion, a plurality of electrodes for detecting an electrocardiographic signal of a human body, a pulse wave sensor configured to detect a pulse wave of the human body, and a vibration sensor configured to detect vibration caused by beating of a heart of the human body, the sensor device being adapted to be attached on an upper arm of the human body and used thereon; and an analysis processing unit configured to calculate a pre-ejection time and a pulse transit time of the heart based on time series data of the electrocardiographic signal, time series data of the pulse wave, and time series data of the vibration caused by the beating of the heart of the human body.

Also, the configurations and processing described above can be combined with one another to constitute the present invention unless the combination leads to contradiction.

### Advantageous Effects of Invention

The present invention can provide a wearable sensor that includes a plurality of biological information sensors and that can be easily attached and detached without burden even by a user who does not have special knowledge, and a technique related to biological information measurement using the wearable sensor.

### BRIEF DESCRIPTION OF DRAWINGS

FIG. 1A is an external perspective view illustrating an outline of a biological information measurement device according to a first embodiment of the present invention.

FIG. 1B is a diagram illustrating an outline of an inner circumferential surface of a belt portion of the biological information measurement device according to the first embodiment of the present invention.

FIG. 2A is a schematic front view illustrating the biological information measurement device according to the first embodiment. FIG. 2B is an explanatory diagram illustrating a placement position of a vibration sensor of the biological information measurement device according to the first embodiment.

FIG. 3 is a block diagram illustrating a functional configuration of the biological information measurement device according to the first embodiment.

FIG. 4 is a diagram illustrating an electrocardiographic waveform obtained from one heartbeat, a waveform of vibration caused by the heartbeat, a pulse wave waveform, and a relationship among indicators that can be calculated from differences between reference points of the waveforms.

FIG. 5 is a flowchart illustrating a flow of blood pressure measurement processing by the biological information measurement device according to the first embodiment.

FIG. 6 is a schematic front view of a biological information measurement device according to a first modified example of the first embodiment.

FIG. 7A is a first explanatory diagram on a second modified example of the first embodiment.

FIG. 7B is a second explanatory diagram on the second modified example of the first embodiment.

FIG. 7C is a third explanatory diagram of the second modified example of the first embodiment.

FIG. 8A is an external perspective view illustrating an outline of a biological information measurement device according to a second embodiment of the present invention.

FIG. 8B is a diagram illustrating an outline of an inner circumferential surface of a belt portion of the biological information measurement device according to the second embodiment.

FIG. 9 is an explanatory diagram illustrating placement positions of vibration sensors of the biological information measurement device according to the second embodiment.

FIG. 10 is a block diagram illustrating a functional configuration of the biological information measurement device according to the second embodiment.

FIG. 11 is a flowchart illustrating a processing flow related to calibration of a blood pressure calculation formula in the biological information measurement device according to the second embodiment.

FIG. 12 is a diagram illustrating an outline of a biological information processing system according to a third embodiment of the present invention.

FIG. 13 is a block diagram illustrating a functional configuration of the biological information processing system according to the third embodiment.

### DESCRIPTION OF EMBODIMENTS

### First Embodiment

Embodiments of the present invention will be specifically described below with reference to the drawings. However, it should be noted that the dimensions, quality of materials, shape, relative arrangement, and the like of the components described in the embodiments below are not intended to limit the scope of this invention to them alone, unless otherwise stated.

### Device Configuration

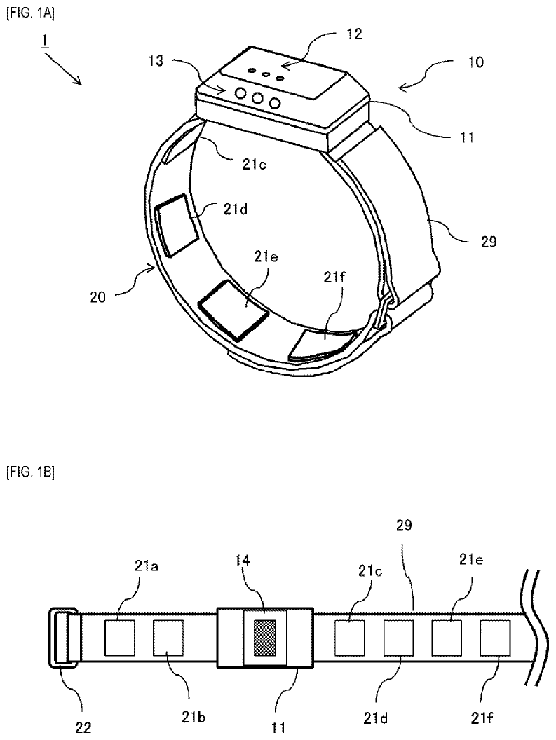

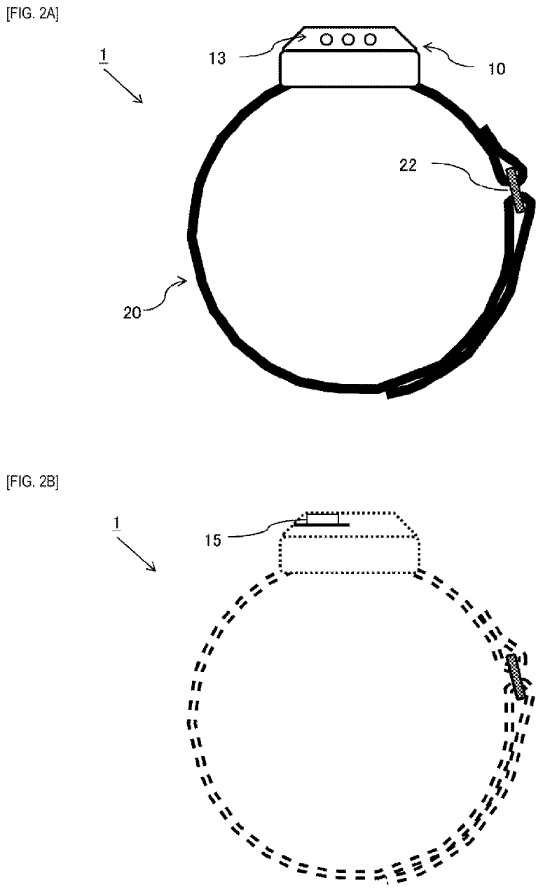

FIGS. 1A and 1B, and FIGS. 2A and 2B are schematic views illustrating a configuration of a biological information measurement device 1 in a first embodiment. FIG. 1A illustrates an external perspective view of the biological information measurement device 1, and FIG. 1B illustrates an outline of an inner circumferential surface of a belt portion 20 of the biological information measurement device 1. Additionally, FIG. 2A illustrates a schematic front view of the biological information measurement device 1, and FIG. 2B is a diagram illustrating a placement position of a vibration sensor 15 in a main body of the biological information measurement device 1.

As illustrated in FIGS. 1A and 1B, and FIGS. 2A to 2B, the biological information measurement device 1 includes a main body portion 10 provided with a main body housing 11, a control unit (not illustrated in FIGS. 1A and 1B), an LED indicator 12, an operation button 13, a pulse wave sensor 14, and the vibration sensor 15, and the like, and a belt portion 20 including a fixing belt 29, an electrode unit of a plurality of electrodes 21a, 21b, 21c, 21d, 21e, 21f, and a belt annular loop 22. Each of the electrodes of the electrode unit is electrically connected to the main body portion via an electrical line (not illustrated) or the like disposed inside the belt portion 20, and a user wears the biological information measurement device 1, for example, on the left upper arm with the fixing belt 29 such that each electrode comes into contact with the skin surface. Additionally, although not illustrated, the fixing belt 29 is provided with a hook-and-loop fastener portion, and one end portion of the fixing belt 29 is passed through the belt annular loop 22 and folded back to be engaged with the hook-and-loop fastener. Thus, the fixing belt 29 is formed into a ring shape and can be fixed to the upper arm.

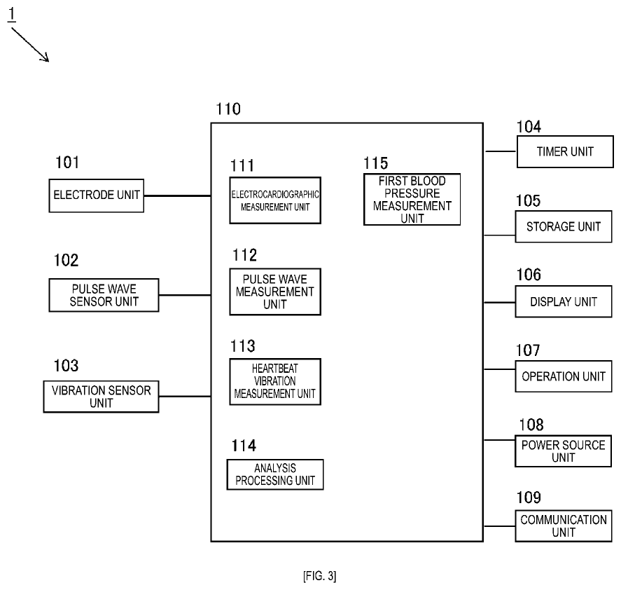

FIG. 3 illustrates a block diagram illustrating a functional configuration of the biological information measurement device 1. As illustrated in FIG. 3, the biological information measurement device 1 includes functional units of a control unit 110, an electrode unit 101, a pulse wave sensor unit 102, a vibration sensor unit 103, a timer unit 104, a storage unit 105, a display unit 106, an operation unit 107, a power source unit 108, and a communication unit 109.

Functional Configuration

The control unit 110 is means serving to control the biological information measurement device 1, and includes, for example, a central processing unit (CPU) and the like. Upon receiving operation of the user via the operation unit 107, the control unit 110 controls each component of the biological information measurement device 1 to execute various types of processing such as biological information measurement and information communication in accordance with a predetermined program. Note that the predetermined program is stored in the storage unit 105 described below to be read therefrom. The control unit 110 further includes, as functional modules, an electrocardiographic measurement unit 111, a pulse wave measurement unit 112, a heartbeat vibration measurement unit 113, an analysis processing unit 114, and a first blood pressure measurement unit 115. These functional modules will be described below.

The electrode unit 101 includes six electrodes 21a, 21b, 21c, 21d, 21e, 21f and functions as a sensor unit configured to detect an electrocardiographic signal. Specifically, in a state where the biological information measurement device 1 is attached, two of the electrodes in an opposing positional relationship pair up with each other, and an electrocardiographic signal is detected based on a potential difference between the two electrodes in a pair. In other words, three types of electrocardiographic signals can be simultaneously detected from three pairs of electrodes.

The pulse wave sensor unit 102 includes a desired pulse wave sensor 14 and functions as a sensor unit configured to detect a pulse wave signal. The pulse wave sensor 14 in the present embodiment is a reflection type photoelectric pulse wave sensor disposed on the lower surface side of the main body housing 11 (i.e., the surface that comes into contact with the skin when worn) as illustrated in FIG. 1B. The reflection type photoelectric pulse wave sensor can detect a blood flow rate (a change in volume of the blood vessels) that changes in accordance with beating of the heart by irradiating a living body with infrared light, red light, or green light and detecting, with the use of a photodiode or the like, the light reflected in the living body.

The vibration sensor unit 103 includes the desired vibration sensor 15, functions as a vibration sensor for detecting vibration caused by the beating of the heart, and acquires BCG data. The vibration sensor 15 in the present embodiment is, for example, a piezoresistive acceleration sensor, and is configured as a micro electro mechanical systems (MEMS) sensor chip. As illustrated in FIG. 2B, in the present embodiment, the vibration sensor 15 is disposed near an inner wall on the upper surface side (i.e., the surface located farthest from the skin surface when worn) in the main body housing 11. When the vibration caused by the beating of the heart is transmitted through the human body (arm) and shakes the device, amplitude increases as a position becomes farther from the skin surface. Therefore, with such a configuration, (the waveform of) the vibration can be easily detected.

Additionally, although not illustrated, each sensor unit including the electrode unit 101 includes an amplifier unit configured to amplify a signal output from the sensor, an A/D conversion unit configured to convert an analog signal into a digital signal, a filter circuit configured to remove a noise component, and the like.

The timer unit 104 functions to measure time with reference to a real time clock (RTC, not illustrated). For example, the time when a predetermined event occurs is counted and output.

The storage unit 105 includes a main storage (not illustrated) such as a random access memory (RAM) and stores various types of information such as application programs and biological information data measured by each measurement unit described below. In addition to the RAM, for example, a long-term storage medium such as a flash memory is provided, and thus long-term biological information can be stored.

The display unit 106 includes the LED indicator 12, and is configured to inform the user of a state of the device, occurrence of a predetermined event, and the like by lighting, blinking, or the like of the LED indicator 12. Additionally, the operation unit 107 includes a plurality of the operation buttons 13, and is configured to receive an input operation from the user via the operation button and function to cause the control unit 110 to execute processing in accordance with the operation.

The power source unit 108 includes a battery (not illustrated) configured to supply power required for operation of the device. The battery may be, for example, a secondary battery such as a lithium ion battery, or may be a primary battery. In a case where a secondary battery is provided, a charging terminal or the like may be provided. The communication unit 109 includes an antenna for wireless communication, a wire communication terminal (both not illustrated), and the like and functions to communicate with another device such as an information processing terminal. Note that the communication unit 109 may also serve as a charging terminal.

The electrocardiographic measurement unit 111 acquires time series data of an electrocardiographic signal from the electrode unit 101, measures an electrocardiographic waveform of the user, and stores ECG data in the storage unit 105. The pulse wave measurement unit 112 acquires time series data of a pulse wave signal from the pulse wave sensor unit 102, measures a pulse wave waveform of the user, and stores PPG data in the storage unit 105. The heartbeat vibration measurement unit 113 acquires time series data of vibration caused by vibration from a heartbeat of the user from the vibration sensor unit 103, generates a ballistocardiogram, and stores BCG data in the storage unit 105.

The analysis processing unit 114 calculates a pre-ejection period (PEP) and a pulse transit time (PTT) of the heart of the user based on the ECG data (time series data of the electrocardiographic signal), the PPG data (time series data of the pulse wave), and the BCG data (time series data of vibration caused by beating of the heart) that are stored in the storage unit 105. Specifically, the PEP and the PTT are calculated by extracting a reference point of the heartbeat (for example, a peak of an R wave in the ECG data, a rising point of a pulse wave in the PPG data, or an ejection time of blood in the BCG data) from each of the ECG data, the PPG data, and the BCG data, and obtaining a time difference between the reference points.

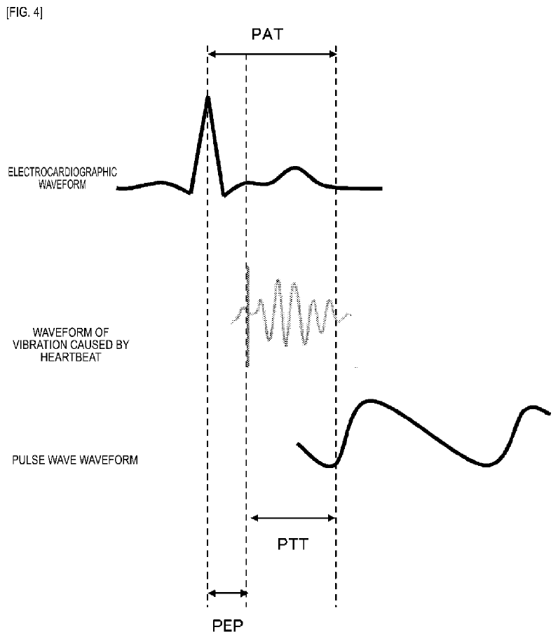

FIG. 4 is a diagram illustrating an electrocardiographic waveform obtained from one heartbeat, a waveform of vibration caused by the heartbeat, a pulse wave waveform, and a relationship among indicators (PAT, PEP, PTT) that can be calculated from differences between reference points of the waveforms. As illustrated in FIG. 4, the PAT can be calculated by obtaining the difference (time) between the reference point of the electrocardiographic waveform and the reference point of the pulse wave waveform. The PEP can be calculated by obtaining the difference (time) between the reference point of the electrocardiographic waveform and the reference point of the vibration waveform. The PTT can be calculated by subtracting the PEP from the PAT. The PTT can also be calculated by obtaining the difference (time) between the reference point of the heartbeat vibration waveform and the reference point of the pulse wave waveform. However, it is easy to detect an R wave of the electrocardiographic waveform, and by using the R wave as a reference for all, each indicator can be obtained easily and accurately.

The first blood pressure measurement unit 115 calculates a blood pressure value of the user for each heartbeat based on the PTT acquired by the analysis processing unit 114 and a blood pressure calculation formula stored in advance in the storage unit 105. Although it has been known for a long time that the pulse transit time and the blood pressure are correlated with each other, there is an individual difference in such a correlation. Therefore, by preparing a relational expression optimized for each user as a blood pressure calculation formula by performing a test in advance and inputting a PTT value into the relational expression, a blood pressure value (for example, SBP) can be calculated.

Flow of Blood Pressure Measurement Processing

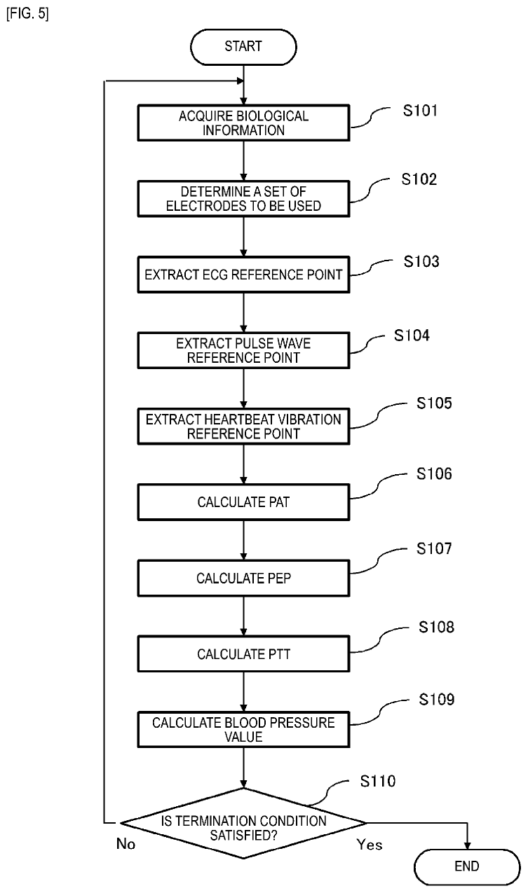

Next, blood pressure measurement processing of the biological information measurement device 1 according to the present embodiment will be described based on FIG. 5. FIG. 5 is a flowchart illustrating a flow of blood pressure measurement processing by the biological information measurement device 1 according to the present embodiment.

Prior to the blood pressure measurement (biological information acquisition), the user wears the biological information measurement device 1, for example, on the left upper arm by using the belt portion 20 such that each electrode of the electrode unit 101 comes into contact with the skin surface. Then, by operating the operation button 13, the blood pressure measurement (biological information acquisition) is started.

Thereafter, each of the electrocardiographic measurement unit 111, the pulse wave measurement unit 112, and the heartbeat vibration measurement unit 113 acquires biological information and stores the biological information in the storage unit 105 (S101). Next, a pair of electrodes to be used of the three pairs of electrodes, more accurately, whether to perform subsequent processing with the use of the ECG data obtained from a potential difference of which pair of electrode is determined by the electrocardiographic measurement unit 111 (S102). In this case, a pair of electrodes from which the most normal (and clear) electrocardiographic waveform can be obtained is selected.

Subsequently, the analysis processing unit 114 performs extraction (S103) of an electrocardiographic waveform reference point, extraction (S104) of a pulse wave waveform reference point, and extraction (S105) of a heartbeat vibration waveform reference point. Further, calculation of the PAT (S106) and calculation of the PEP (S107) are performed based on the extracted reference points, and calculation of the PTT (S108) is performed based on the calculated PAT and PEP. Furthermore, by using the calculated PTT, the first blood pressure measurement unit 115 calculates a blood pressure value (S109), and the blood pressure measurement for one heartbeat ends. Note that data of the measured blood pressure is stored as time series data in the storage unit 105.

Then, processing for determining whether a predetermined measurement termination condition (for example, an end button is pressed, storage capacity is not sufficiently left, or the like) is satisfied is performed (S110). Here, when it is determined that the termination condition is not satisfied, processing returns to step S101, and the subsequent processing is repeated. On the other hand, when it is determined in step S110 that the termination condition is satisfied, the blood pressure measurement ends.

In addition, when the blood pressure value calculated in step S109 satisfies a predetermined condition (for example, the calculated value deviates from predetermined upper and lower limit values) or when the time series data of blood pressure values stored in the storage unit 105 satisfies a predetermined condition (for example, the blood pressure equal to or higher than a predetermined value continues over the predetermined time), the LED indicator 12 is lighted or blinked and thus the user may be notified.

According to the biological information measurement device 1 according to the present embodiment as described above, a wearable device can be provided that can be easily attached and detached even by the own user without special knowledge and can non-invasively calculate a blood pressure value for each heartbeat. As a result, continuous blood pressure measurement can be easily performed at all times (or for a long period of time) without deteriorating the quality of the daily life of the user, which can help with early detection of a disease or of a sign thereof

### First Modified Example

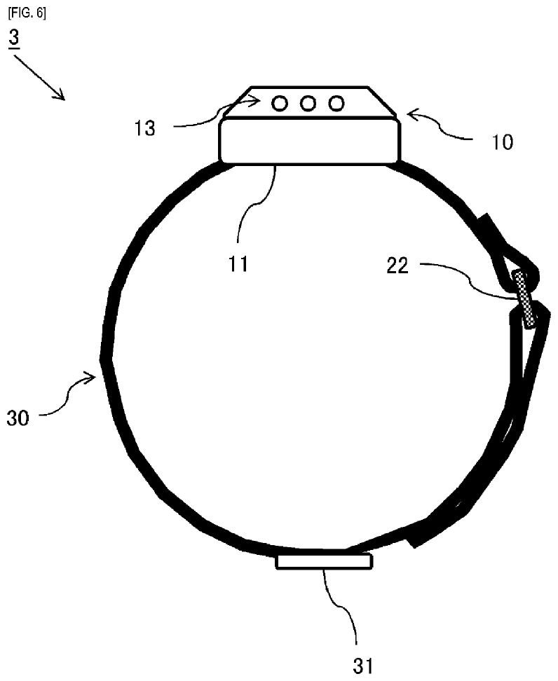

The biological information measurement device 1 according to the first embodiment described above can be modified in various ways. FIG. 6 illustrates an example of such a modification. In the modified examples and other embodiments described below, the same components and processing as those in the first embodiment are denoted by the same reference numerals, and detailed description thereof will be omitted. FIG. 6 is a schematic front view of a biological information measurement device 3 according to a first modified example. The biological information measurement device 3 according to the present modified example has substantially the same configuration as that of the biological information measurement device 1, but is different in that a plurality of vibration sensors are provided.

The biological information measurement device 3 includes a vibration sensor housing 31 at a position opposed to the main body housing 11 in the circumferential direction of a belt portion 30. A second vibration sensor (not illustrated) is disposed in the vibration sensor housing 31. Further, the second vibration sensor is electrically connected to the main body portion 10 via a conductive portion (not illustrated) in the belt portion 30. Furthermore, in a state where the biological information measurement device 3 is attached on the upper arm of a user, the main body housing 11 and the vibration sensor housing 31 are disposed at positions opposed to each other in the circumferential direction of the upper arm, and the vibration sensor 15 and the second vibration sensor form a pair located in the opposite direction with the upper arm interposed therebetween.

In the case of a wearable device that is constantly worn and continuously measures body information, the sensor is compressed and vibration is attenuated depending on the posture of a wearer (for example, in the supine position at bedtime), which may cause a situation where vibration with appropriate intensity cannot be detected. In this regard, as in the biological information measurement device 3 according to the present modified example, a plurality of vibration sensors are disposed at positions opposed to each other with the arm interposed therebetween, and thus at least one of the sensors can detect appropriate vibration.

### Second Modified Example

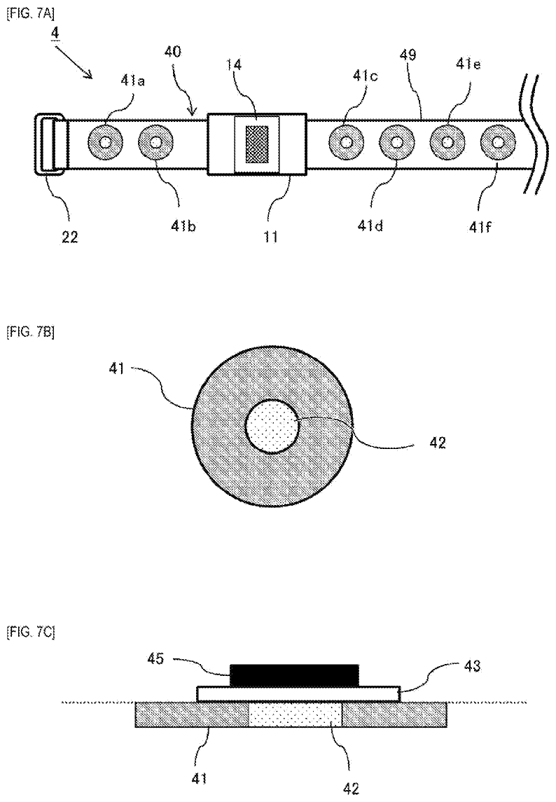

Next, another modified example will be described. FIGS. 7A to 7C are diagrams illustrating a biological information measurement device 4 according to a second modified example. FIG. 7A is a diagram illustrating an outline of an inner circumferential surface of a belt portion 40 of the biological information measurement device 4. FIG. 7B is a schematic bottom view of an electrode 41 of the biological information measurement device 4. FIG. 7C is a simplified cross-sectional view illustrating the electrode and a vibration sensor 45 of the biological information measurement device 4. Note that a broken line in FIG. 7C indicates a line of the inner circumferential surface of a fixing belt 49, and a portion above the broken line indicates a position inside the fixing belt 49.

In the biological information measurement device 4 according to the present modified example, the vibration sensor 45 is a microphone, and the electrode 41 and the vibration sensor 45 are integrally formed. Additionally, as illustrated in FIG. 7A, electrodes 41a, 41b, 41c, 41d, 41e, 41f formed integrally with the vibration sensor 45 are disposed on the inner circumferential surface of the belt portion 40 of the biological information measurement device 4.

Next, the configuration of the electrode 41 according to the present modified example will be described in more detail. As illustrated in FIG. 7B and FIG. 7C, the electrode 41 of the biological information measurement device 4 is configured such that a substrate 43 and the vibration sensor 45 mounted on the substrate are disposed on the surface opposite to a contact surface of the electrode. With such a configuration, the substrate for acquiring potential information of the electrode and the substrate on which the vibration sensor is disposed can be integrated with each other; therefore, the device members can be omitted.

Further, the electrode 41 is formed in a circular shape and is provided with a hollow portion extending through the center portion in a thickness direction. The hollow portion serves as a sound pickup structure of a microphone as the vibration sensor 45. The electrode and a function to amplify the vibration detected by the microphone are integrally configured as just described, and thus an S/N ratio of a signal acquired by the vibration sensor can be improved while the number of components is reduced.

Furthermore, a conductive resin 42 having hardness comparable with that of the human skin is filled in the hollow portion of the electrode 41 so as to be flush with the contact surface of the electrode 41 that contacts the skin. The vibration is acquired through the resin having hardness comparable with that of the skin, and thus transit efficiency of the vibration is improved, and the S/N ratio of the acquired signal can be improved. In addition, since the resin is conductive, a reduction in contact resistance in the contact surface of the electrode in which the contact area with the skin is reduced by providing the hollow portion can be suppressed.

### Second Embodiment

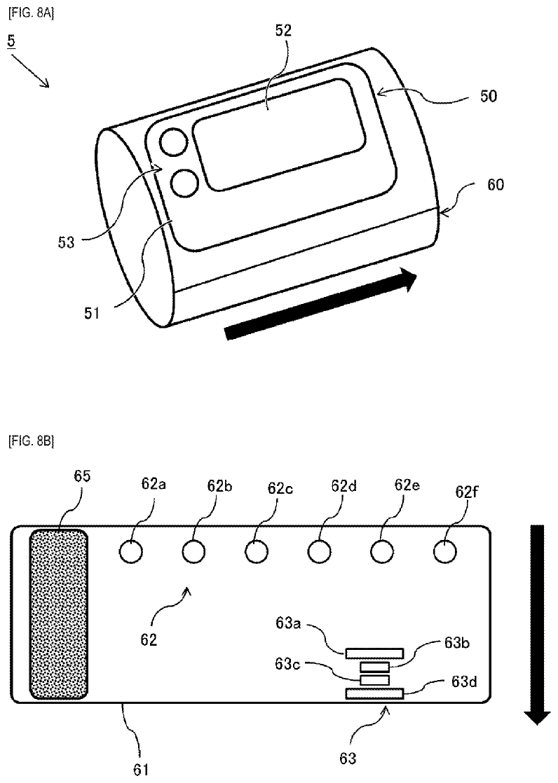

Next, another embodiment of the present invention will be described based on FIGS. 8A and 8B to FIG. 11.

Device Configuration

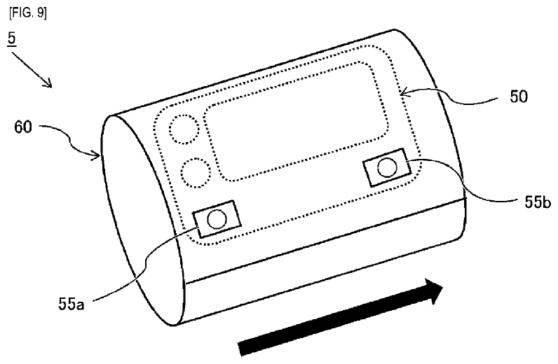

FIGS. 8A and 8B, and FIG. 9 are schematic views illustrating a configuration of a biological information measurement device 5 according to a second embodiment. FIG. 8A illustrates an external perspective view of the biological information measurement device 5. FIG. 8B illustrates an outline of an inner circumferential surface of a belt portion 60 of the biological information measurement device 5. In addition, FIG. 9 is a diagram illustrating placement positions of vibration sensors 55a, 55b in a main body housing 51 of the biological information measurement device 5. The black arrows in FIGS. 8A and 8B, and FIG. 9 indicate the direction in which the periphery side is located when the biological information measurement device 5 is attached on the upper arm of a human body. In other words, the side indicated by the arrows is the periphery side (the side farther from the heart) when the device is worn.

As illustrated in FIGS. 8A and 8B, and FIG. 9, the biological information measurement device 5 generally includes a main body portion 50 and the belt portion 60. The main body portion 50 includes the main body housing 51, a control unit (not illustrated in FIGS. 8A and 8B, and FIG. 9), a liquid crystal display 52, an operation button 53, the vibration sensors 55a, 55b, and the like. In addition, the belt portion 60 includes a fixing belt 61, an electrocardiographic electrode unit 62 including a plurality of electrodes 62a, 62b, 62c, 62d, 62e, 62f, a pulse wave electrode unit 63 including a plurality of electrodes 63a, 63b, 63c, 63d, and a hook-and-loop fastener (hook portion 65). In the present embodiment, the electrodes 63a, 63b, 63c, 63d correspond to a pulse wave sensor.

As illustrated in FIG. 8B, the electrocardiographic electrode unit 62 and the pulse wave electrode unit 63 are placed such that the pulse wave electrode unit 63 is located closer to the periphery side when the device is worn. When obtaining the pulse transit time from the ECG or PCG (or BCG), as a pulse wave detection position becomes farther from the central side of the human body, that is, as the pulse transit time becomes longer, data with a higher S/N ratio can be acquired. Therefore, the pulse sensor is placed closer to the periphery side as described above, and thus an accurate pulse transit time can be easily calculated.

Each of the electrodes is electrically connected to the main body portion 50 via an electrical line (not illustrated) or the like disposed in the belt portion 60, and a user wears the biological information measurement device 5, for example, on the left upper arm by using the fixing belt 61 such that each electrode comes into contact with the skin surface. Although not illustrated, a loop portion of a hook-and-loop fastener is disposed on the outer circumferential surface side of the fixing belt 61, and the fixing belt 61 is worn around the upper arm and the hook portion 65 is brought into engagement with the loop portion. Thus, the device can be fixed to the upper arm.

Further, as illustrated in FIG. 9, the biological information measurement device according to the present embodiment includes two vibration sensors (the vibration sensors 55a, 55b), and the vibration sensors are placed respectively near both ends of the main body portion 50 in the longitudinal direction. In other words, the vibration sensors are placed at an interval from a side close to the periphery side of the user to a side close to the central side in a state where the device is attached on the upper arm. With such a configuration, a noise component can be removed (reduced) by comparing signals output from a plurality of sensors having different transit distances of vibration. Furthermore, the vibration sensors 55a, 55b are disposed on separate substrates separated from each other. Such a configuration is desirable in order to remove (reduce) the noise component. This is because when the sensors are integrated by a rigid body such as a substrate even though the sensors themselves are separately placed, the same vibration data (signal) is acquired.

Functional Configuration

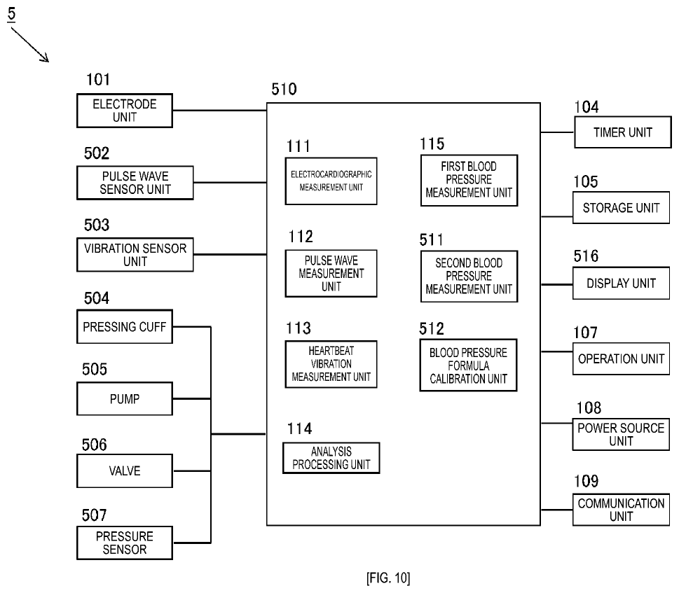

FIG. 10 is a block diagram illustrating a functional configuration of the biological information measurement device 5. As illustrated in FIG. 10, the biological information measurement device 5 includes functional units of a control unit 510, the electrode unit 101, a pulse wave sensor unit 502, a vibration sensor unit 503, a pressing cuff 504, a pump 505, a valve 506, a pressure sensor 507, the timer unit 104, the storage unit 105, a display unit 516, the operation unit 107, the power source unit 108, and the communication unit 109.

Of the functional units, the electrode unit 101, the timer unit 104, the storage unit 105, the operation unit 107, the power source unit 108, and the communication unit 109 are configured in the same manner as those of the biological information measurement device 1 according to the first embodiment, and thus description thereof will be omitted. In addition, the display unit 516 is also the same as the display unit 106 of the biological information measurement device 1 in terms of a functional aspect except that the display unit includes the liquid crystal display 52 in the present embodiment and thus can display a variety of information.

As illustrated in FIG. 8B, the pulse wave sensor unit 502 in the present embodiment includes the four electrodes 63a, 63b, 63c, 63d serving as a pulse wave sensor. The pulse wave sensor unit 502 detects pulse waves by feeding a current between the electrodes 63a and 63d and detecting a voltage between the electrodes 63b and 63c in an energized state. When the electrodes 63a and 63d are in an energized state, changes in electrical impedance (changes in arterial volume) due to pulse waves transiting through the arteries in contact with the electrodes 63b and 63c can be detected, and thus pulse waves can be detected.

The pressing cuff 504, the pump 505, the valve 506, and the pressure sensor 507 are all components used for blood pressure measurement by an oscillometric method as described below. The pressing cuff 504 is an air bag disposed in the belt portion 60, and air flows into and out of the pressing cuff 504 by opening and closing of the pump 505 and the valve 506 that are controlled by a second blood pressure measurement unit 511 described below. The pressure sensor 507 detects pressure in the pressing cuff 504 and generates an electrical signal indicating the pressure. The pressure sensor 507 can serve, for example, as a piezoresistive pressure sensor. The pump 505 and the valve 506 are disposed in the main body portion 50.

The control unit 510 is means that serves to control the biological information measurement device 5, and is configured substantially in the same manner as that of the biological information measurement device 1 according to the first embodiment. However, there is a difference in that the second blood pressure measurement unit 511 and a blood pressure calculation formula calibration unit 512 are provided as functional modules.

When a predetermined condition is satisfied, the second blood pressure measurement unit 511 controls the pressing cuff 504 (the pump 505 and the valve 506), performs blood pressure measurement by the oscillometric method based on an output signal of the pressure sensor 507, and stores the measurement result in the storage unit 105. Here, examples of the predetermined condition may include, in addition to a case where an instruction input of the user via the operation button 53 is received, a case where the measured blood pressure value of the first blood pressure measurement unit 115 deviates from the upper and lower limit values over a predetermined time, a case where a fluctuating range within a predetermined time is equal to or greater than a predetermined value, and the like.

When blood pressure measurement is performed by the second blood pressure measurement unit 511 (that is, by the oscillometric method), the blood pressure calculation formula calibration unit 512 uses the result of the blood pressure measurement stored in the storage unit 105 and thereby calibrates the blood pressure calculation formula for calculating blood pressure by the first blood pressure measurement unit 115.

Flow of Blood Pressure Calculation Formula Calibration Processing

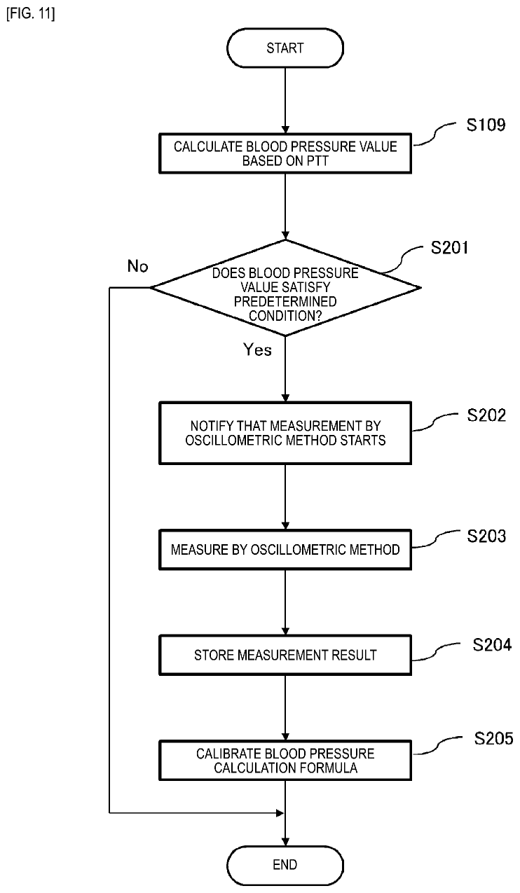

Hereinafter, a flow of processing related to calibration of the blood pressure calculation formula of the biological information measurement device 5 according to the present embodiment will be described based on FIG. 11. FIG. 11 is a flowchart illustrating a flow of processing related to calibration of the blood pressure calculation formula in the biological information measurement device 5. As illustrated in FIG. 11, in measurement of a blood pressure value by the device, first, continuous measurement for each heartbeat based on the PTT is performed by the first blood pressure measurement unit 115 (S109) as is the case with the first embodiment.

Thereafter, the control unit 510 performs processing for determining whether the measured blood pressure value by the first blood pressure measurement unit 115 satisfies a predetermined condition (S201). Examples of the predetermined condition may include a case where the measured value deviates from the upper and lower limit values over a predetermined time, a case where a fluctuating range of the measured value within a predetermined time is equal to or greater than a predetermined value, and the like. Here, when it is determined that the predetermined condition is not satisfied, the blood pressure calculation formula is not calibrated, and the flow is temporarily terminated.

On the other hand, when it is determined in step S201 that the predetermined condition is satisfied, the control unit 510 notifies the user via the liquid crystal display 52 that blood pressure measurement by the oscillometric method is to be performed (S202). In addition to the display, notification may be performed by voice. After performing the notification to the user, the second blood pressure measurement unit 511 controls the pump 505 and the valve 506 to perform blood pressure measurement by the oscillometric method (S203). When the blood pressure measurement by the oscillometric method is finished, the measurement result is stored in the storage unit 105 (S204). Additionally, in this case, the measurement result may be displayed on the liquid crystal display 52.

Then, the blood pressure calculation formula is calibrated by the blood pressure calculation formula calibration unit 512 based on the measurement result of the blood pressure stored in the storage unit 105 in step S204, the updated blood pressure calculation formula is stored in the storage unit 105 (S205), and the series of flow of the blood pressure calculation formula calibration is temporarily finished. Additionally, after the notification to the user is performed in processing of step S202 described above, processing may proceed to step S203 upon reception of permission (measurement instruction) from the user.

According to the biological information measurement device 5 of the present embodiment as described above, continuous blood pressure measurement for each heartbeat based on the PTT and the highly accurate blood pressure measurement by the oscillometric method can be attained with one wearable device. As a result, only by wearing one device, the user can selectively use the simple continuous blood pressure measurement and the accurate blood pressure measurement depending on the situation. In addition, since the blood pressure calculation formula based on the PTT can be calibrated by using the blood pressure measurement result by the oscillometric method, high accuracy can be maintained also in the continuous blood pressure measurement for each heartbeat.

### Third Embodiment

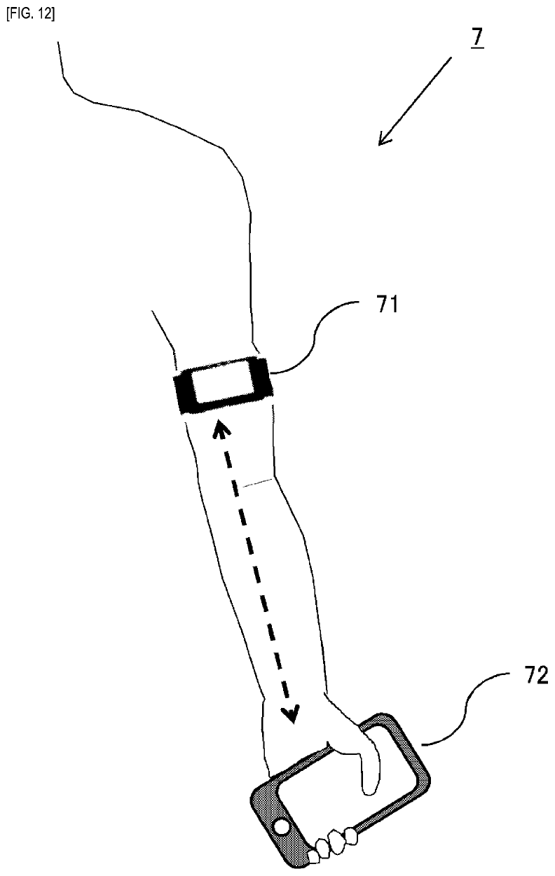

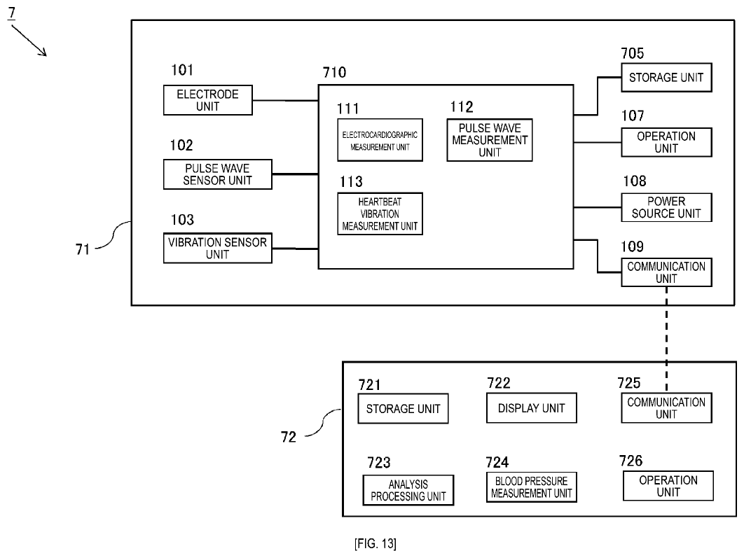

In each of the examples described above, the present invention is applied as a biological information measurement device, and all of the functions, including the storage unit and the display unit, are integrated into one biological information measurement device. However, the present invention can be applied as a biological information processing system in which a part of such configuration and functions are separated. FIG. 12 and FIG. 13 illustrate examples of such an information processing system. FIG. 12 illustrates an outline of a biological information processing system 7 according to the present embodiment. As illustrated in FIG. 12, the biological information processing system 7 includes a sensor device 71 to be attached on the upper arm of a user and an information processing terminal 72 configured to process biological information acquired by the sensor device 71. Although not illustrated, the sensor device 71 is a wearable device including a plurality of electrodes (an electrocardiographic sensor), a pulse wave sensor, and a vibration sensor, and is used while being fixed to the upper arm of the user with a belt or the like. The information processing terminal 72 may be of any type as long as the terminal can communicate with the sensor device 71. For example, a smartphone can be used as the information processing terminal 72.

FIG. 13 is a block diagram illustrating a functional configuration of the sensor device 71 and the information processing terminal 72 of the biological information processing system 7. The sensor device 71 includes functional units of the electrode unit 101, the pulse wave sensor unit 102, the vibration sensor unit 103, a control unit 710, a storage unit 705, the operation unit 107, the power source unit 108, and the communication unit 109. Further, the control unit 710 includes, as its functional modules, the electrocardiographic measurement unit 111, the pulse wave measurement unit 112, and the heartbeat vibration measurement unit 113.

The sensor device 71 includes many configurations similar to those of the biological information measurement device 1 of the first embodiment, but is particularly different in that the functional module in the control unit 710 is omitted and the display unit is omitted. Furthermore, the storage unit 705 includes only a main storage device such as a RAM or a ROM and has limited storage capacity. Therefore, the biological information measured by each sensor unit is transmitted to the information processing terminal 72 via the communication unit 109 in real time as described below.

The information processing terminal 72 communicates with the sensor device 71 via a communication unit 725 and receives the biological information of the user measured by the sensor device 71. The communication standard is not particularly limited, but communication can be performed by a wireless communication standard such as Bluetooth (registered trademark), Wi-Fi (registered trademark), or infrared communication. Note that the hardware configuration of the information processing terminal 72 is the same as the configuration of a smartphone. For example, a touch panel display serves as both a display unit 722 and an operation unit 726.

The information received via the communication unit 725 is stored in a storage unit 721, and analysis processing by an analysis processing unit 723 and blood pressure measurement processing by a blood pressure measurement unit 724 are performed based on the stored information. Note that the analysis processing unit 723 and the blood pressure measurement unit 724 are respectively configured to have the same functions as those of the analysis processing unit 114 and the first blood pressure measurement unit 115 of the biological information measurement device 1, and thus description thereof is omitted.

As described above, the biological information processing system 7 of the present embodiment is configured such that sensing of biological information is performed by the sensor device 71 and analysis processing of the biological information, blood pressure measurement processing, and the like are performed by the information processing terminal 72. According to such a configuration, the configuration of the wearable device can be simplified. In addition, since an existing information processing terminal can be utilized as in the embodiments described above, costs of the entire system can be reduced.

Other Points

The description of each example described above is merely illustrative of the present invention, and the present invention is not limited to the specific embodiments described above. Within the scope of the technical idea of the present invention, various modifications and combinations may be made. For example, the photoelectric pulse wave sensor adopted in the first embodiment may be applied to the device of the second embodiment, or the integrated configuration of the electrode and the vibration sensor adopted in the second modified example of the first embodiment may be applied to the device of the second embodiment. Further, the vibration sensors disposed at two positions in the second embodiment may be replaced with only the vibration sensor disposed on the central side.

Furthermore, in each of the examples described above, each piece of biological information and each indicator are used to measure a blood pressure value. However, the biological information and the indicator themselves may be utilized. For example, since it is assumed that the longer the PEP is, the more the heart function is decreased, when the PEP exceeds a predetermined value, a warning thereof may be issued.

### REFERENCE NUMERALS LIST

1, 3, 4, 5 Biological information measurement device
10, 50 Main body portion
11, 51 Main body housing
12 LED indicator
13, 53 Operation button
14 Pulse wave sensor
15, 45 Vibration sensor
101 Electrode unit
102 Pulse wave sensor unit
103 Vibration sensor unit
104 Timer unit
105, 705, 721 Storage unit
106, 516, 722 Display unit
107, 726 Operation unit
108 Power source unit
109, 725 Communication unit
110, 510, 710 Control unit
20, 30, 40, 60 Belt portion
21a, 21b, 21c, 21d, 21e, 21f, 41a, 41b, 41c, 41d, 41e, 41f, 62a, 62b, 62c, 62d, 62e, 62f,
63a, 63b, 63c, 63d Electrode
22 Belt annular loop
29, 49, 61 Fixing belt
31 Vibration sensor housing
42 Conductive resin
43 Substrate
52 Liquid crystal display
62 Electrocardiographic electrode unit
63 Pulse wave electrode unit
65 Hook portion
7 Biological information processing system
71 Sensor device
72 Information processing terminal

What is claimed is:

1. A biological information measurement device adapted to be attached on an upper arm of a human body and used thereon, the biological information measurement device comprising:
a belt portion adapted to be wound around the upper arm;
electrocardiographic measurement unit, the unit including a plurality of electrodes for detecting an electrocardiographic signal of the human body;
pulse wave measurement unit including a pulse wave sensor for detecting a pulse wave of the human body;
heartbeat vibration measurement unit including a vibration sensor for detecting vibration caused by beating of a heart of the human body; and
an analysis processing unit configured to calculate a pre-ejection time and a pulse transit time of the heart based on time series data of the electrocardiographic signal, time series data of the pulse wave, and time series data of the vibration caused by the beating of the heart.

2. The biological information measurement device according to claim 1, further comprising:
a first blood pressure measurement unit configured to calculate a blood pressure value of the human body based on the pulse transit time calculated by the analysis processing unit.

3. The biological information measurement device according to claim 2, further comprising:
a pressing cuff;
a fluid supply configured to supply a fluid to the pressing cuff;
a pressure sensor configured to detect pressure in the pressing cuff; and
a second blood pressure measurement unit configured to calculate a blood pressure value of the human body based on an output signal of the pressure sensor.

4. The biological information measurement device according to claim 3,
wherein the first blood pressure measurement unit performs, based on the blood pressure value measured by the second blood pressure measurement unit, calibration of a calculation formula for calculating a blood pressure value based on the pulse transit time.

5. The biological information measurement device according to claim 1,
wherein the pulse wave sensor is disposed to be located on a side closer to a periphery of the human body than the plurality of electrodes, in a state where the biological information measurement device is attached on the upper arm.

6. The biological information measurement device according to claim 1, comprising:
a housing in which at least the vibration sensor is housed,
wherein the vibration sensor is housed at a location that is near an inner wall surface of the housing and is located farthest from the skin surface of the human body, in a state where the biological information measurement device is attached on the upper arm.

7. The biological information measurement device according to claim 1,
wherein the vibration sensor includes a plurality of vibration sensors, and the plurality of vibration sensors are disposed at intervals from a side close to a periphery of the human body to a side close to a center of the human body, in a state where the biological information measurement device is attached on the upper arm.

8. The biological information measurement device according to claim 7,
wherein the vibration sensor is a sensor mounted on a substrate, and the plurality of vibration sensors are mounted on different substrates separated from each other.

9. The biological information measurement device according to claim 1,
wherein the vibration sensor includes a plurality of vibration sensors, and the plurality of vibration sensors include at least one set of vibration sensors disposed at positions opposed to each other in a circumferential direction of the upper arm, in a state where the biological information measurement device is attached on the upper arm.

10. The biological information measurement device according to claim 1,
wherein the vibration sensor is disposed to be located near an end portion on a side close to a center of the human body, in a state where the biological information measurement device is attached on the upper arm.

11. The biological information measurement device according to claim 1,
wherein at least one of the plurality of electrodes and the vibration sensor are integrally formed, and
the vibration sensor is disposed on a side opposed to a contact surface of the electrode integrally formed with the vibration sensor, the contact surface contacting the human body.

12. The biological information measurement device according to claim 11,
wherein the vibration sensor is a microphone, and
the electrode integrally formed with the vibration sensor is provided with a sound pickup structure.

13. The biological information measurement device according to claim 12,
wherein the sound pickup structure is a hollow portion disposed to extend through the electrode in a thickness direction.

14. The biological information measurement device according to claim 13,
wherein the hollow portion is filled with a resin having a hardness comparable with a hardness of human skin to be flush with the contact surface.

15. The biological information measurement device according to claim 14,
wherein the resin is a conductive resin.

16. A biological information processing system comprising:
a sensor device including a belt portion, a plurality of electrodes for detecting an electrocardiographic signal of a human body, a pulse wave sensor configured to detect a pulse wave of the human body, and a vibration sensor configured to detect vibration caused by beating of a heart of the human body, the sensor device being adapted to be attached on an upper arm of the human body and used thereon; and
an analysis processing unit configured to calculate a pre-ejection time and a pulse transit time of the heart based on time series data of the electrocardiographic signal, time series data of the pulse wave, and time series data of the vibration caused by the beating of the heart of the human body.

17. The biological information measurement device according to claim 2, wherein the pulse wave sensor is disposed to be located on a side closer to a periphery of the human body than the plurality of electrodes, in a state where the biological information measurement device is attached on the upper arm.

18. The biological information measurement device according to claim 3, wherein the pulse wave sensor is disposed to be located on a side closer to a periphery of the human body than the plurality of electrodes, in a state where the biological information measurement device is attached on the upper arm.

19. The biological information measurement device according to claim 4, wherein the pulse wave sensor is disposed to be located on a side closer to a periphery of the human body than the plurality of electrodes, in a state where the biological information measurement device is attached on the upper arm.

20. The biological information measurement device according to claim 2, comprising:
a housing in which at least the vibration sensor is housed,
wherein the vibration sensor is housed at a location that is near an inner wall surface of the housing and is located farthest from the skin surface of the human body, in a state where the biological information measurement device is attached on the upper arm.

21. The biological information measurement device according to claim 3, comprising:
a housing in which at least the vibration sensor is housed,
wherein the vibration sensor is housed at a location that is near an inner wall surface of the housing and is located farthest from the skin surface of the human body, in a state where the biological information measurement device is attached on the upper arm.

22. The biological information measurement device according to claim 4, comprising:
a housing in which at least the vibration sensor is housed,
wherein the vibration sensor is housed at a location that is near an inner wall surface of the housing and is located farthest from the skin surface of the human body, in a state where the biological information measurement device is attached on the upper arm.

23. The biological information measurement device according to claim 5, comprising:
a housing in which at least the vibration sensor is housed,
wherein the vibration sensor is housed at a location that is near an inner wall surface of the housing and is located farthest from the skin surface of the human body, in a state where the biological information measurement device is attached on the upper arm.

24. The biological information measurement device according to claim 2,
wherein the vibration sensor includes a plurality of vibration sensors, and the plurality of vibration sensors are disposed at intervals from a side close to a periphery of the human body to a side close to a center of the human body, in a state where the biological information measurement device is attached on the upper arm.

25. The biological information measurement device according to claim 3,
wherein the vibration sensor includes a plurality of vibration sensors, and the plurality of vibration sensors are disposed at intervals from a side close to a periphery of the human body to a side close to a center of the human body, in a state where the biological information measurement device is attached on the upper arm.

26. The biological information measurement device according to claim 4,
wherein the vibration sensor includes a plurality of vibration sensors, and the plurality of vibration sensors are disposed at intervals from a side close to a periphery of the human body to a side close to a center of the human body, in a state where the biological information measurement device is attached on the upper arm.

27. The biological information measurement device according to claim 5,
wherein the vibration sensor includes a plurality of vibration sensors, and the plurality of vibration sensors are disposed at intervals from a side close to a periphery of the human body to a side close to a center of the human body, in a state where the biological information measurement device is attached on the upper arm.

28. The biological information measurement device according to claim 2, wherein the vibration sensor includes a plurality of vibration sensors, and the plurality of vibration sensors include at least one set of vibration sensors disposed at positions opposed to each other in a circumferential direction of the upper arm, in a state where the biological information measurement device is attached on the upper arm.

29. The biological information measurement device according to claim 3,
wherein the vibration sensor includes a plurality of vibration sensors, and the plurality of vibration sensors include at least one set of vibration sensors disposed at positions opposed to each other in a circumferential direction of the upper arm, in a state where the biological information measurement device is attached on the upper arm.

30. The biological information measurement device according to claim 4,
wherein the vibration sensor includes a plurality of vibration sensors, and the plurality of vibration sensors include at least one set of vibration sensors disposed at positions opposed to each other in a circumferential direction of the upper arm, in a state where the biological information measurement device is attached on the upper arm.

31. The biological information measurement device according to claim 5,
wherein the vibration sensor includes a plurality of vibration sensors, and the plurality of vibration sensors include at least one set of vibration sensors disposed at positions opposed to each other in a circumferential direction of the upper arm, in a state where the biological information measurement device is attached on the upper arm.

32. The biological information measurement device according to claim 2,
wherein the vibration sensor is disposed to be located near an end portion on a side close to a center of the human body, in a state where the biological information measurement device is attached on the upper arm.

33. The biological information measurement device according to claim 3,
wherein the vibration sensor is disposed to be located near an end portion on a side close to a center of the human body, in a state where the biological information measurement device is attached on the upper arm.

34. The biological information measurement device according to claim 4,
wherein the vibration sensor is disposed to be located near an end portion on a side close to a center of the human body, in a state where the biological information measurement device is attached on the upper arm.

35. The biological information measurement device according to claim 5,
wherein the vibration sensor is disposed to be located near an end portion on a side close to a center of the human body, in a state where the biological information measurement device is attached on the upper arm.

36. The biological information measurement device according to claim 2,
wherein at least one of the plurality of electrodes and the vibration sensor are integrally formed, and
the vibration sensor is disposed on a side opposed to a contact surface of the electrode integrally formed with the vibration sensor, the contact surface contacting the human body.

37. The biological information measurement device according to claim 3,
wherein at least one of the plurality of electrodes and the vibration sensor are integrally formed, and
the vibration sensor is disposed on a side opposed to a contact surface of the electrode integrally formed with the vibration sensor, the contact surface contacting the human body.

38. The biological information measurement device according to claim 4,
wherein at least one of the plurality of electrodes and the vibration sensor are integrally formed, and
the vibration sensor is disposed on a side opposed to a contact surface of the electrode integrally formed with the vibration sensor, the contact surface contacting the human body.

39. The biological information measurement device according to claim 5,
wherein at least one of the plurality of electrodes and the vibration sensor are integrally formed, and
the vibration sensor is disposed on a side opposed to a contact surface of the electrode integrally formed with the vibration sensor, the contact surface contacting the human body.
